(19) United States
(12) Plant Patent Application Publication
(10) Pub. No.: US 2003/0154526 P1
(43) Pub. Date: Aug. 14, 2003

(54) Shrub rose plant named 'Lady Elsie May'

(76) Inventor: Reinhard Noack, Gutersloh (DE)

Correspondence Address: Bruce M. Monroe, RATNER & PRESTIA, P.O. Box 7228, Wilmington, DE, 19803, US

(21) Appl. No.: 10/075,873

(22) Filed: Feb. 13, 2002

(51) Int. Cl. A01H 5/00

(52) U.S. Cl. PLT/107

(57) ABSTRACT

A new and distinct rose variety of the shrub class which is particularly well suited for growing as an attractive ornamentation to the landscape, characterized by its continuous and abundant production of long-lasting brilliant orange-pink flowers, its upright, dense and bushy growth habit, its attractive glossy foliage that contrasts well with the brilliant orange-pink blooms, and its outstanding disease resistance.

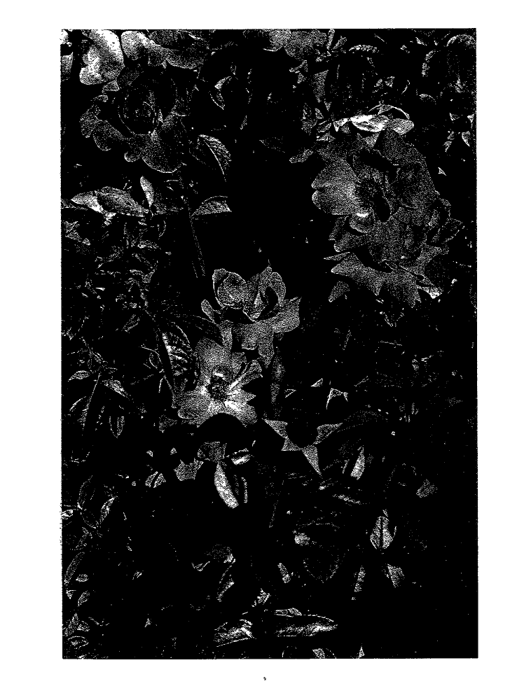

### FIELD OF THE INVENTION

This invention relates to a new and distinct variety of rose plant of the shrub rose class.

### SUMMARY OF THE INVENTION

The present invention relates to a new and distinct variety of rose plant of the shrub class, which is particularly well suited for growing as an attractive ornamentation to the landscape. The varietal denomination of this new and distinct variety of rose plant is ‘Lady Elsie May.’

By cross pollination, I have produced a new and improved rose variety which is distinguished from all other rose varieties of which I am aware, as evidenced by the following unique combination of characteristics which are outstanding therein:

(1) Its continuous and abundant production of long-lasting brilliant orange-pink flowers.

(2) Its upright, dense and bushy growth habit.

(3) Its attractive glossy foliage that contrasts well with the brilliant orange-pink blooms.

(4) Its outstanding disease resistance.

Asexual reproduction of this new variety by budding was first performed in Gütersloh, Germany. Asexual reproduction of this new variety in Kent County, on the eastern shore of Maryland, shows that the foregoing and all other characteristics come true to form and are established and transmitted through successful propagation.

### BRIEF DESCRIPTION OF THE ILLUSTRATION

The accompanying illustration shows typical specimens of the vegetative growth and flower of this new variety in different stages of development, depicted in color as nearly true as it is reasonably possible to make the same in a color illustration of this character.

### DETAILED DESCRIPTION OF THE NEW VARIETY

The following is a detailed description of my new rose cultivar with color descriptions using terminology in accordance with a Royal Horticulture Society Colour Chart (RHSCC), except where ordinary dictionary significance of color is indicated.

Parentage: Unknown

Class: Shrub

The following observations are made of one-year old specimens grown outdoors in Kent County, Maryland, during the month of September.

### FLOWER

Blooming habit: Recurring, continuous.

A. Bud:

Size.—Small. About ½ to ¾ inch long when the petals start to unfurl.

Form.—Long and pointed.

Sepals.—Curling back as the bud opens. Length — about ¾ inch to 1 inch. There are three normally appendaged sepals. There are two unappendaged sepals. Shape — triangular, pointed with slight serration at tip. Texture — slightly hairy. Color: Upper side — Green Group 139B. Underside — Greyed Green Group 193D.

Color.—Red Group 53B when sepals start to unfurl.

Peduncle.—Length — 1 to ½ inch long. Straight. Aspect — smooth. Color — Greyed Red Group 182A.

Calyx tube.—Length — about ¼ inch. Aspect — smooth. Color — Green Group 141C.

B. Bloom:

Size.—Average open size is about 3 inches.

Borne.—In clusters and in large sprays.

Stems.—Short, typically about 3 to 6 inches.

Form.—Initially cup-shaped, flattening on maturity.

Fragrance.—Rose fragrance.

C. Petals:

Size.—Length: typically about 1½ inches. Width: typically about 1½ inches.

Shape.—Triangular. Margins entire with a slight indentation near the center of the outer margin.

Texture.—Smooth.

Arrangement.—Symmetrically arranged in a circular outline.

Number.—Under normal conditions typically about 10-11, with 1 to 3 petaloids in the center.

Color.—Orange-pink. When first opened, the upper side is close to Red Group 46C. The under side is close to Red Group 52B.

Color change.—As the bloom ages the edge of the upper side of the petals tends to Red Purple Group 66A. The underside tends to Red Purple Group 57C.

Persistence.—Drop off cleanly.

Lasting quality.—On the plant: Long, at least 10-12 days.

### REPRODUCTIVE PARTS

A. Reproductive parts:

Stamens.—Length — about ½ inch.

Anthers.—Size: Medium, about ⅛ inch. Quantity: Many, about 80. Color: Yellow-Orange, near Yellow-Orange Group 22-A. Arrangement: Regular around styles.

Filaments.—Color: Red near Red Group 39A.

Pollen.—Color: Greyed Orange Group 163A.

Styles, stigmas, and pistils.—None observed. Flower contains a continuous disc, Yellow Green Group 151C.

B. Hips: None observed.

C. Seeds: None observed.

### PLANT

A. Form: Upright.

B. Growth: Very vigorous, uniform branching on main stems, new growth occurs regularly throughout season. Height obtained in first season — about 1½ to 2 feet. Mature Plant: Height — about 3 feet. Width — about 3 ft.

C. Foliage: Compounded 3 to 5 leaflets on single bloom, typically 5 leaflets on main canes.

Size.—Leaflets — ¾ to ½ inches long and ½ to 1 inch wide.

Quantity.—Abundant, covers plant to ground.

Arrangement.—Alternate; Uniform and equally spaced.

Color.—New foliage: Upper side — near to Green Group 137A; under side — near Green Group 137C. Old foliage: upper side — Green Group 139A, under side — Yellow Green Group 147B.

Shape.—Oval with slight point at tip.

Venation.—Parallel.

Texture.—Upper side — leathery, glossy. Under side — smooth between ribs.

Edge.—Serrated.

Serration.—Very small and uniform.

Petiole.—Color: Upper side — Greyed-Purple Group 183A. Under side — Yellow-Green Group 146D. Texture — Smooth. Length — ¾ to 1¾ inches.

Rachis.—Color — Yellow-Green Group 144B. Texture — Smooth.

Stipules.—Shape — Pointed. Texture — Medium smooth. Color: Yellow-Green Group — 146A. Length — about ½ inch. Width at top. — about ¼ inch.

Resistance to disease.—Blackspot — Outstanding. Mildew Outstanding. Rust — not observed.

D. Wood:

New wood.—Color — Greyed-Orange Group 177A. Bark — smooth.

Old wood.—Color — Green Group 138A. Bark — smooth.

E. Thorns and Prickles

Thorns.—Quantity — ordinary. On laterals from stalk — few. Shape — pointed and curved down. Form — flat base. Length — about ⅛ to ¼ inch. Color (when young) — Greyed-Purple Group 185A. Position — irregular.

Prickles.—Quantity — few. On laterals — very few. Size — typically less than ⅛ inch. Shape — pointed. Position — irregular.

F. Winter hardiness: Excellent. Hardy to zone 4.

I claim:

1. A new and distinct variety of rose plant of the shrub class which is particularly well suited for growing as an attractive ornamentation to the landscape, substantially as herein shown and described, characterized particularly as to novelty by its continuous and abundant production of long-lasting brilliant orange-pink flowers, its upright, dense and bushy growth habit, its attractive glossy foliage that contrasts well with the brilliant orange-pink blooms, and its outstanding disease resistance.
